(19) United States
(12) Patent Application Publication
(10) Pub. No.: US 2016/0187313 A1
(43) Pub. Date: Jun. 30, 2016

(54) Core Sample Analysis System with Conditioned Environment

(71) Applicant: GE Energy Oilfield Technology, Inc., Houston, TX (US)

(72) Inventors: Thomas G. Szudajski, Houston, TX (US); John C. Boot, Atlanta, GA (US)

(21) Appl. No.: 14/587,247

(22) Filed: Dec. 31, 2014

(51) Int. Cl. G01N 33/24; G01N 23/00

(52) U.S. Cl. CPC G01N 33/24; G01N 23/00

(57) ABSTRACT

A system for analyzing a cote sample from a wellbore, where the analysis takes place in the field and proximate the wellbore. The system includes a trailer with a unit for scanning the core sample and obtaining information within the sample. The unit is housed in a cabinet having a cabinet interior that is maintained at a set temperature and humidity with conditioned air. The cabinet also has sidewalls that define a pressure barrier between the inside of the cabinet and an isolation space that is between the cabinet and inner surface of the trailer. The unit includes a manipulator system for moving the core sample through a rotating scan source in the scanning unit. A sample enclosure provides a barrier between the ambient zone surrounding the sample and the isolation space in the cabinet.

### BACKGROUND OF THE INVENTION

1. Field of Invention

The present disclosure relates in general to a method and system for analyzing a core sample from a wellbore. More specifically, the present disclosure relates to a method and system for evaluating a core sample from a wellbore with computerized tomography.

2. Description of Prior Art

Various techniques are currently in use for identifying the presence of hydrocarbons in subterranean formations. Some techniques employ devices that emit a signal from a seismic source, and receive reflections of the signal on surface. Others involve disposing logging devices downhole in a wellbore intersecting the subterranean formation, and interrogating the formation from within the wellbore. Example downhole exploration devices include seismic tools that can transmit and receive seismic signals, or ones that simply receive a seismic signal generated at surface. Other devices collect and sample fluid from within the formation, or from within the wellbore. Nuclear tools are also employed that direct radiation into the formation, and receive radiation that scatters from the formation. Analyzing the scattered radiation can provide information about fluids residing in the formation adjacent the wellbore, the type of fluid, and information about other materials next to the wellbore, such as gravel pack.

Logging downhole also is sometimes done while the wellbore itself is being drilled. The logging devices are usually either integral with a drill bit used during drilling, or on a drill string that rotates the drill bit. The logging devices typically are either nuclear, seismic, can in some instances optical devices. In some instances a core is taken from the wellborn and analyzed after being retrieved to the surface. Analyzing the core generally provides information about the porosity and/or permeability of the rock formation adjacent the wellbore. Cores are generally elongated cylindrical members and obtained with a coring tool having an open barrel for receiving and retaining the core sample.

### SUMMARY OF THE INVENTION

Disclosed herein is an example of a system for analyzing a core sample and which includes a system for analyzing a core sample, where the system includes a mobile enclosure, a cabinet in the mobile enclosure having sidewalls which define a cabinet interior that is selectively at a higher pressure than an environment ambient to the mobile enclosure, a core sample scan system within the cabinet, and a loading assembly coupled to the core sample scan system and that selectively moves the core sample within the core sample scan system. The system can further include an isolation space within the mobile enclosure that has sidewalls that define a barrier, so that the isolation space is selectively at a higher pressure than the environment ambient to the mobile enclosure, In an example, the cabinet interior is selectively at a higher pressure than the isolation space. In an example, the corn sample scan system includes a core carries that selectively receives the core sample and that is reciprocated within an annular sample enclosure. A space can be included inside in the sample enclosure that is in communication with the environment ambient to the mobile enclosure so that the sample enclosure defines a portion of a pressure barrier between the environment ambient to the mobile enclosure and the cabinet interior. In one embodiment, included is an isolation space within the mobile enclosure that is between the cabinet interior and the space inside the sample enclosure, wherein the isolation space is selectively at a higher pressure than the environment ambient to the mobile enclosure. A brush seal is optionally included that has a curved outer periphery in sealing contact with an inner surface of the sample enclosure; and which defines a pressure barrier between the environment ambient to the mobile enclosure and the cabinet interior. The mobile enclosure can include a popout section that selectively projects laterally outward into a deployed configuration. Further optionally provided is a membrane having an end coupled with the popout section and an opposite end mounted on a roller, so that when the popout section projects into the deployed configuration, the membrane unrolls from the roller and defines a pressure barrier between the environment ambient to the mobile enclosure and the cabinet interior. The system can include a flooring section on a lower inside surface of the popout section made up of sections that are laid down when die popout section is the deployed configuration, wherein the flooring section defines a pressure barrier between the environment ambient to the mobile enclosure and the cabinet interior.

Also disclosed, herein is a system for analyzing a core sample that is made up of a mobile enclosure, an isolation space within the mobile enclosure and having sidewalls that define a barrier, so that the isolation space is selectively at a higher pressure than the environment ambient to the mobile enclosure. The system also includes a cabinet in the mobile enclosure having sidewalls which define a cabinet interior that is selectively at a higher pressure than pressure in the isolation space, an annular sample enclosure that projects into the cabinet a core sample scan system within the cabinet having a core carrier that selectively receives the core sample and that is reciprocated within the annular sample enclosure, and a loading assembly coupled to the core sample scan system and that selectively moves the core sample within the core sample scan system. Optionally included is a space inside in the sample enclosure that is in communication with the environment ambient to the mobile enclosure so that the sample enclosure defines a portion of a pressure barrier between the environment ambient to the mobile enclosure and the cabinet interior. The system can include a brush seal that couples to and circumscribes the core carrier, the brush seal having a curved outer periphery that is in sealing contact with an inner surface of the sample enclosure and which defines a pressure barrier between the environment ambient to the mobile enclosure and the cabinet interior. A popout section can be provided on the mobile enclosure that selectively projects laterally outward into a deployed configuration, also optionally included is a membrane having mi end coupled with the popout section and an opposite end mounted on a roller, so that when the popout section projects into the deployed configuration, the membrane unrolls from the roller and defines a pressure barrier between the environment ambient to the mobile enclosure and the cabinet interior. The system may farther have a flooring section on a lower inside surface of the popout section with sections that are laid down when the popout section is the deployed configuration, wherein the flooring section defines a pressure barrier between the environment ambient to the mobile enclosure and the cabinet interior.

A method of analyzing a com sample is also included herein and that includes inserting the core sample within a core scans system that is housed within a mobile enclosure, scanning the core sample, and pressurizing a cabinet that encloses the core scan system to a pressure that exceeds a pressure of an environment that is ambient to the mobile enclosure. The method can further involve forming an isolation space in the mobile enclosure that surrounds the cabinet, and pressurizing the isolation space to a pressure that exceeds the pressure of the environment that is ambient to the mobile enclosure. The method may further optionally include supplying a constant flow of conditioned air to the cabinet at designated conditions that satisfies operational requirements of components within the cabinet.

### BRIEF DESCRIPTION OF DRAWINGS

Some of the features and benefits of the present invention having been stated, others will become apparent as the description proceeds when taken in conjunction with the accompanying drawings, in which:

FIG. 1 is a plan partial sectional view of an example of a system for analyzing a core sample.

FIG. 2 is an overhead view of an example of a cabinet for shielding radiation and conditioning a scanning unit tor a core sample.

FIG. 3 is an axial sectional view of the cabinet of FIG. 2 and taken along lines 3-3,

FIG. 4 is a perspective view of the cabinet of FIG. 2.

FIG. 5 is a perspective view of the cabinet of FIG. 2 in partial phantom view and an example scanning unit in the cabinet.

FIGS. 6A and 6B are schematic views of a trailer with a scan unit in a cabinet, and showing examples of conditioned air zones within the trailer,

FIG. 7 is a perspective view of an example of the trailer of FIG. 2, with a popout portion in. a deployed configuration.

FIG. 8 is a sectional view of the trailer of FIG. 7 taken along lines 8-8.

While the invention, will be described in connection with the preferred embodiments, it will be understood that it is not intended to limit the invention to that embodiment. On the contrary, it is intended to cover all alternatives, modifications, and equivalents, as may be included within the spirit and scope of the invention as defined by the appended claims.

### DETAILED DESCRIPTION OF INVENTION

The method and system of the present disclosure will sow be described more fully hereinafter with reference to the accompanying drawings in which embodiments are shown. The method and system of the present disclosure may be in many different forms and should not be construed as limited to the illustrated embodiments set forth herein; rather, these embodiments are provided so that this disclosure will be thorough and complete, and will fully convey its scope to those skilled in the art. Like numbers refer to like elements throughout. In an embodiment, usage of the term “about” includes, but is not necessarily limited to, +/−5% of the cited magnitude. In an embodiment, usage of the term “substantially” includes, but is not necessarily limited to, +/−5% of the cited magnitude.

It is to be further understood that the scope of the present disclosure is not limited to the exact details of construction operation, exact materials, or embodiments shown and described, as modifications and equivalents will be apparent to one skilled in the art. In the drawings and specification, there have been disclosed illustrative embodiments and, although specific terms are employed, they are used in a generic and descriptive sense only and not for the purpose of limitation.

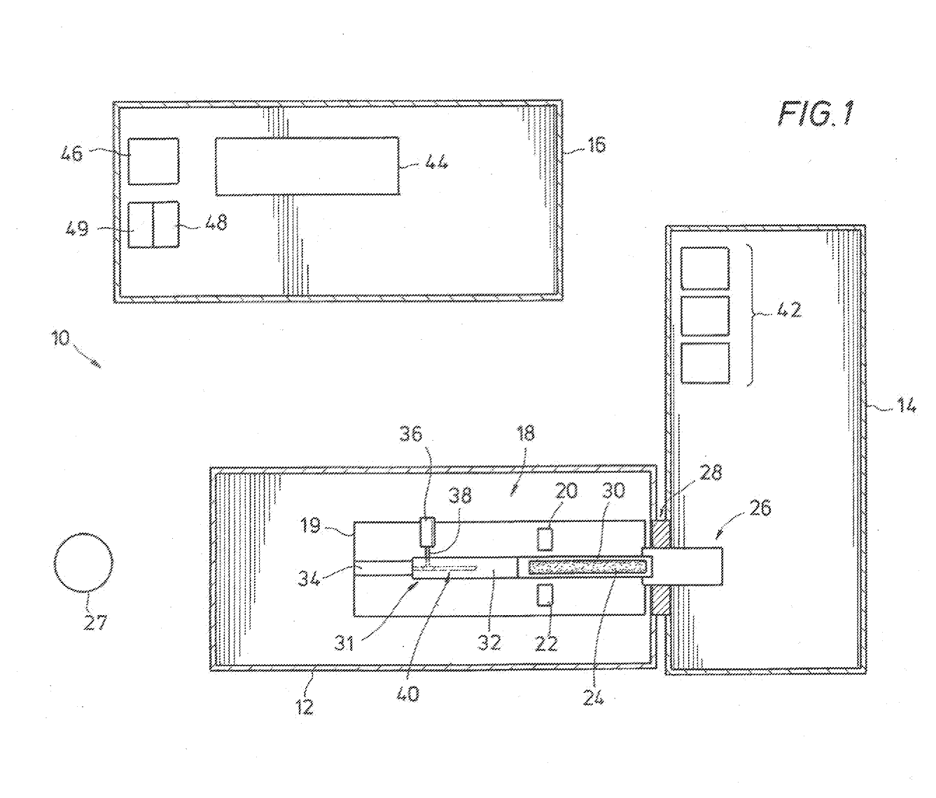

Shown in a plan partial sectional view in FIG. 1 is one sample of a core analysis system 10, which includes first, second and third mobile enclosures. In the example of FIG. 1, the first mobile enclosure is a scan trailer 12, the second mobile enclosure is a handling trailer 14, and the third mobile enclosure is an analysis trailer 16. In one example, each of the enclosures may be part of a tractor trailer system and which are movable by a tractor rig. Examples exist where the mobile enclosures are each mounted on a chassis fitted with wheels (not shown), so that a tractor rig can transport the mobile enclosure to a designated locate, such as to a drilling site. Optionally, each trailer 12, 14, 16 is a container that is offshore and ISO certified. Schematically illustrated in the scan trailer 12 is a scan system 18, and substantially all of which is housed within a cabinet 19. In the illustrated example, cabinet 19 is specially designed to shield any radiation within, generated, inherent or otherwise, from making its way to outside of the cabinet 19. Thus, cabinet 19 is in compliance with 21 C.P.R, 1020.40. Further shown in cabinet 19 is a scan source 20, which in one embodiment includes a device for emitting radiation, such as bat not limited to an X-ray, micro-wave, millimeter wave, etc. A scan receiver 22 is also shown provided within cabinet 19 and combined with scan source 20, in one example, forms a Computed Tomography (CT) scanner.

An elongate and cylindrical core sample 24 is shown axiaily inserted within scan system 18. Core sample 24 is disposed into scan system 18 through a loading assembly 26, which m shown coupled to one end of the scan system 18 and projecting through an opening in a side wall of handling trailer 14. In an example, core sample 24 is taken from a subterranean formation, below system 10, and is retrieved via a wellborn 27 shown adjacent system 10. Thus the wellbore 27 intersects the subterranean formation. Embodiments exist where the system 10 is “onsite” in the field and where the distance between the wellbore 27 to system 10 can range from less than one hundred yards up to five miles, and any distance between. Accordingly, real time analysis while drilling the wellbore 21 can take place within the system 10. Feedback from the analysis can be used by the drilling operator to make adjustments or changes to the drilling operation.

A hatch assembly 28 is schematically illustrated which provides the coupling interlace between trailers 12, 14 and includes sealing around the loading assembly 26. While in scan system 18, core sample 24 rests on a core carrier 30. In an example, core carrier 30 is fabricated from a material transparent to X-Rays, and can support the load of the core sample 24 with minimum deflection to maintain the resolution of a stationary scanner. Core carrier 30 is part of a manipulator system 31, which farther includes a manipulator arm 32 that telescopingly moves along a manipulator base 34. As shown, an end of manipulator arm 32 distal from manipulator base 34 couples onto an end of core carrier 30, so that core carrier is basically cantilevered on an end of the manipulator arm 32. Manipulator arm 32 is shown in m extended position over manipulator base 34. Manipulator arm 32 axially moves with respect to manipulator base 34 via a motor 36 shown having a shaft 38 that couples to manipulator arm 32. In one example, motor 36 is a linear direct current motor. A gear (not shown) on an end of shaft 38 distal from motor 36 engages a gear rack 40 that is provided on manipulator arm 32. Accordingly, selectively operating motor 36 urges manipulator arm 32, core carrier 30 and core sample 24 in an axial direction with respect to scan source 20. Moving manipulator arm 32 into a retracted position, onto manipulator base 34 positions the entire length of core sample 24 in scan system 18, so that all of cure sample 24 may be analyzed by the scan system 18. In one example, the scan source 20 and scan receiver 22 orbit around the core sample 24 and so that when in combination of axial movement of core sample 24 within system 18, a helical scan is taken of core sample 24. Further optionally, motor 36, or additional motors not shown, may manipulate and selectively move manipulator arm vertically and/or laterally to thereby better position core sample 24 into a designated orientation and/or spatial position during the scanning process.

Further shown, in FIG. 1 are a series of work surfaces 42 provided within handling trailer 14. In one example of operation, before or alter core sample 24 is scanned, it may be broken into sections for further analysis and analyzed on surfaces 42. Examples of the surfaces 42 include a crusher, sample divider, and mortar grinder. Additional analysis may take place within analysis trailer 16. Schematically illustrated within analysis trailer 16 are a variety of analysis equipment such as, but not limited to, scanners and spectrometers. One such, analysis equipment is a nanotom 44, which can include a scanning system for scanning the internals of core sample 24, or parts of the core sample. Further analysis equipment in the analysis trailer 16 may be a laser induced spectroscope 46, a Raman spectroscope 48, and near infrared spectroscope 49. It will be understood that alternate embodiments may include more trailers or fewer trailers. For example, an appropriately sized sears system 18 may allow loading assembly 26 to be in scan trailer 12 without projecting through an opening in the trailer and without a hatch assembly 28. A further embodiment may provide work surfaces 42 in the same trailer as the analysis equipment, or the analysis equipment may be contained in handling trailer 14. In yet a further embodiment, scan system 18, loading assembly 26, work surfaces 42 and analysis equipment (e.g., nanotom 44, spectroscopes 46; 48, 49, or others) axe all contained in one trailer.

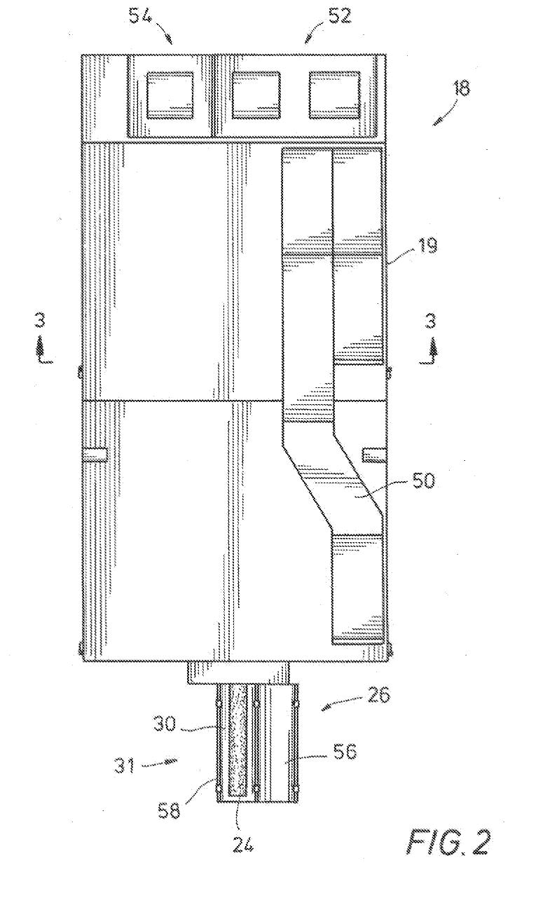

Referring now to FIG. 2, shown in an overhead view is an example of the scan system 18 and an upper surface of cabinet 19. Further illustrated In this example is a conditioning vent 50 on an upper end of the cabinet 19, where conditioning vent 50 provides a path for airflow and that is used in conditioning the inside of the cabinet 19, while blocking the leakage of any radiation from cabinet 19. An advantage of the conditioning vent 50 is that conditioned air at proper temperature and humidity may be injected into the inside of cabinet 19 so that the sensitive devices housed within the cabinet 19 may be maintained in proper operating conditions to ensure normal operating functionality. In an example, operational conditions require maintaining a substantially constant temperature within the cabinet 19. In care embodiment, the temperature variation in the cabinet 19 is kept of within 2 degrees C. of a designated temperature. An advantage of the device described herein is that the temperature in the cabinet 19 can be maintained within the designated range in spite of substantial air -replacement. Air replacement in the cabinet 19, due to the loading mechanism operation, maintains temperature uniformity across the scanner frame and rotary element. In one example, the volumetric rate of air replacement is at least about 4 m³/min. A power distribution panel 52 is shown provided at an aft end of cabinet 19, and which includes buses (not shown) and other devices for distributing power through cabinet 19 into scan system 18. A control panel 54 is shown adjacent power distribution panel 52 and includes hardware and software tor managing control of the operation of the systems house within cabinet 19. Projecting outward past the forward end of cabinet 19 is the loading assembly 26 in an open configuration. In the illustrated example, the loading assembly 26 includes a loading cover 56 and loading basin 58, where the loading cover 56 is shown swung open from a loading basin 58. As shown the core sample 24 has been Inserted into open loading assembly 26 and onto the core carrier 30. As will be described in more detail, below, safety features are included with the system that prevent operation of the manipulator system 31 when the loading assembly 26 is in the open position of FIG. 2.

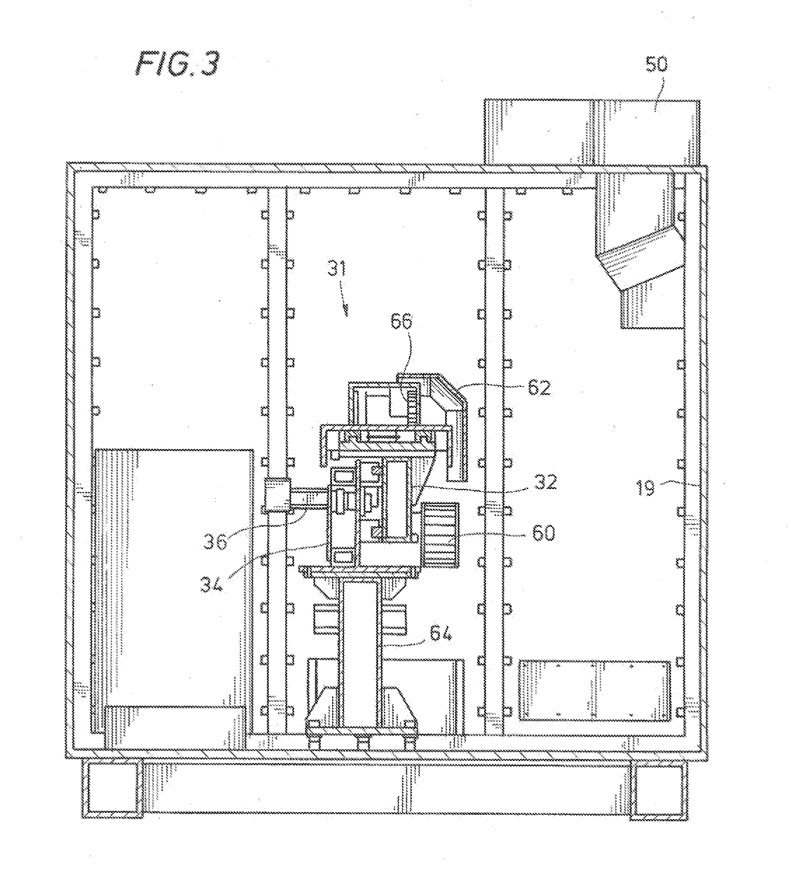

FIG. 3 shows an example of the cabinet 19 in a sectional view and taken along lines 3-3 of FIG. 2. This view which is taken along the axial portion of manipulator system 31 shows one example of a wiring track 60; which has cross members for organizing the control and power wires needed for use in the scan system 18 and as the manipulator arm 32 axially moves with respect to manipulator base 34. Wiring track 60 maintains the wires In a designated location and position with use of wiring track 60 during operation of the manipulator system 31. Further in the example of FIG. 3 is a shroud 62 shown mounted on an upper end of manipulator system 31 and which covers a portion of the upper end and shields components within the manipulator system 31. Manipulator base 34 (and thus manipulator arm 32) is supported on a vertical mounting pedestal 64, which has a generally rectangular cross section along its axis, and has a lower end mounted on the floor of cabinet 19. Shown housed within shroud 62 is a wiring bus 66 which extends axially along the manipulator assembly.

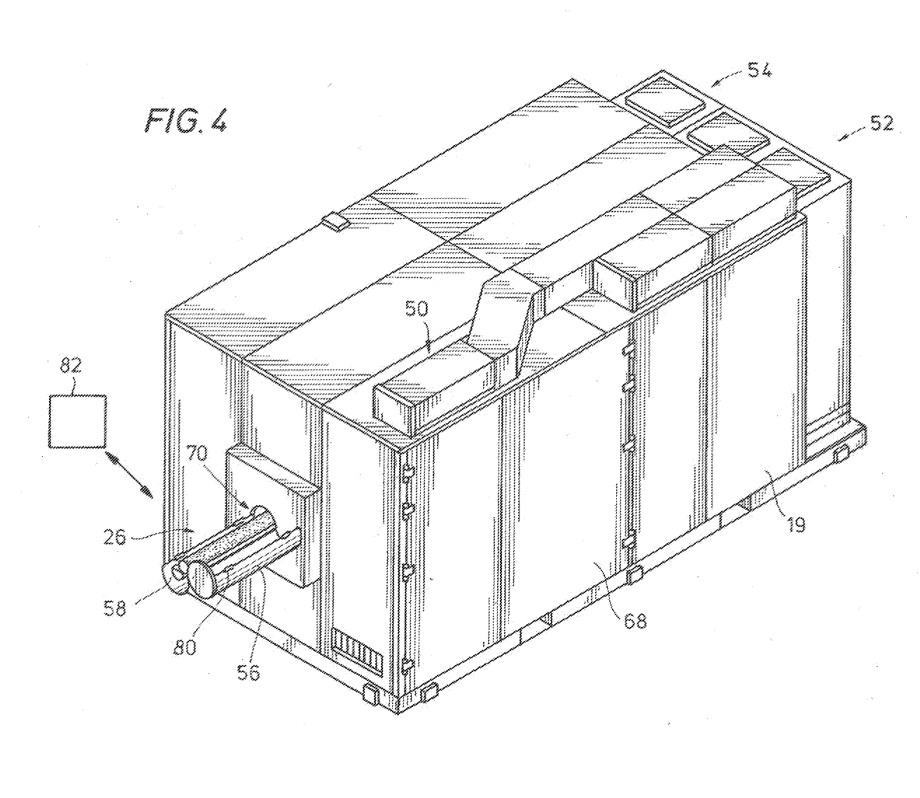

FIG. 4 provides in perspective view of one example of the cabinet 19 and having hinged panel 68 along its outer surface. As indicated above, the structure of cabinet 19 is in compliance with 21 C.F.R., 1020.40. Thus proper protective shielding and interlocking is provided in the panel 68 and along the hinged interlace. An additional safety feature is a door assembly 70 which includes a barrier (not shown) that slides axially across the opening shown at the base of the loading assembly 26 and In a forward wall of cabinet 19. The barrier thus provides a radiation shield from the inside to the outside of cabinet 19 while still allowing core sample loading In compliance with 21 C.F.R. §1020.40.

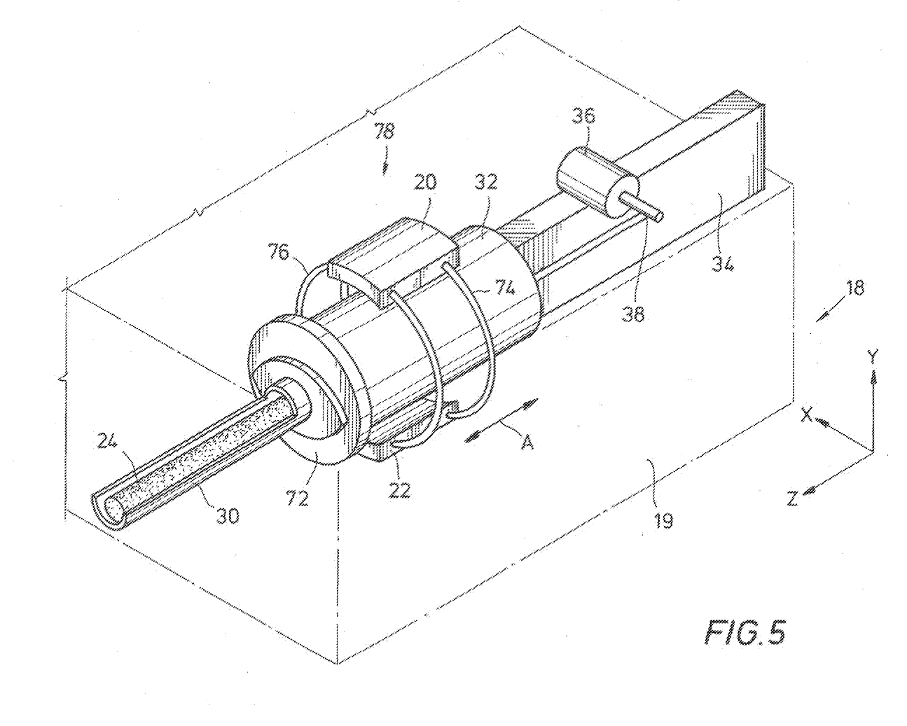

An example of the manipulator assembly within cabinet 19 is illustrated in perspective view in FIG. 5, and where cabinet 19 is shown in a partial phantom view. In this embodiment, a rearward end of manipulator base 34 is supported on a rearward end of cabinet 19; manipulator base 34 extends axially away from, the rearward wall of cabinet 19 with the manipulator arm 32 axially sliding on manipulator base 34. Motor 36 is shown oriented generally perpendicular to an axis of manipulator arm 32 and manipulator base 34, and couples to manipulator arm 32 by shaft 38. Further illustrated is how the core carrier 30 couples to a mounting plate 72; where mounting plate 72 is a generally circular and planar member that mounts on a forward end of manipulator arm 32. In one embodiment, this member along with an extended tunnel provides the seal that inhibits excessive air flow during the loading process.

Axial movement, as shown by the double headed arrow A, of core sample 24 is accomplished via motor 36. X, Y, and Z axes are illustrated to define an example coordinate system for the purposes of reference herein. While not limited to this coordinate system, the axes depict axial movement of any object, such as the core sample 24, to be along the Z axis, vertical movement to be along the Y axis, and lateral movement to be along the X axis, As indicated above, operation of motor 36 can move core sample 24 along all of these axes. Further shown in FIG. 5 arc curved supports 74, 76 that circumscribe manipulator arm 32 and provide a mounting surface for scan source 20 and scan, receiver 22. The combination of the support 74, 76 define a gantry 78 that when rotated puts the scan source 20 and scan receiver 22 at an orbiting rotation around the core sample 24 and provides the scanning capabilities of the scan system 18. As indicated above, the air replacement capabilities provided with cabinet 19 maintains a substantially constant temperature across the gantry 78.

Referring back to FIG. 4, an interlock connector SO is shown, provided on the loading cover 56 and. loading basin 58. The interlock connectors 80 thus may recognize when the cover 56 is in the open position of FIG. 4 and in combination with controller 82 may prevent operation of the manipulator assembly. However, the control system associated with the scan system 18 that allows for motion of the manipulator assembly when the cover 56 is in the closed position and interlock connectors are adjacent one another.

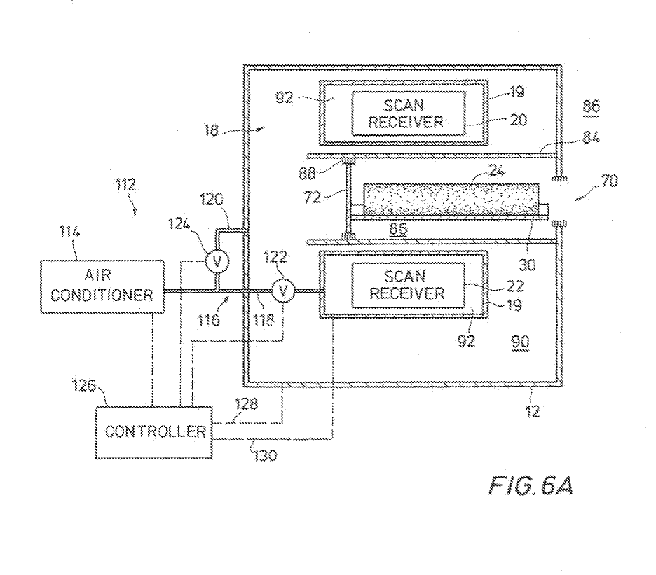

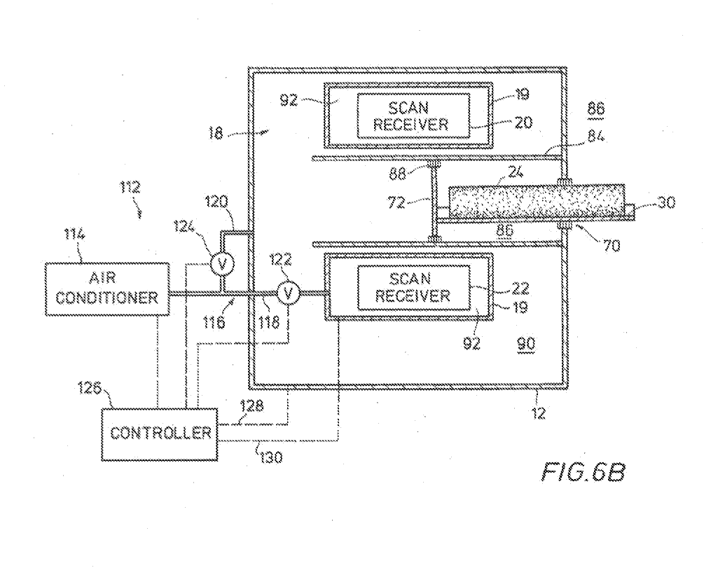

Shown in FIGS. 6A, and 6B is a schematic view of an example of the scan system 18 in the scan trailer 12. In the illustrated example, the core sample 24, core carrier 30, and mounting plate 72 are disposed within an annular sample enclosure 84. An end of sample enclosure 84 is shown up against an inner surface of a sidewall of scan trailer 12 and circumscribing door assembly 70. An opposite end. of sample enclosure 84 is disposed within scan trailer 12 and distal from door assembly 70. As explained in more detail below, the sample enclosure 84 provides a barrier between an ambient zone 86 and inside the scan trader 12. In the example of FIGS. 6A and 6B, ambient zone 86 is illustrated as a volume of space around the scan trailer 12, and which is in communication with the space immediately around the core sample 24. The temperature, pressure, humidity, and particulate level of the ambient acme 86 may vary, thus an advantage exists to isolate the scan system 18 from these varying conditions that can damage or otherwise affect the operation of the seats system 18. A brush seal 88 is shown provided along an outer periphery of the mounting plate 72, which provides sealing capability between mounting plate 72 and an inner surface of sample enclosure 84 and blocks communication between ambient zone 86 and an isolation space 90. Moreover, brush seal 88 maintains a seal against the inner surface of sample enclosure 84 as core sample 24 is fully within scan trader 12 (FIG. 6A), and when core sample 24 is partially within scan trailer 12 (FIG. 6B).

As illustrated in FIGS. 6A and 6B, isolation space 90 is the volume of space between the inner surface of scan trailer 12 and outer surface of cabinet 19. Further shown in the example embodiments of FIGS. 6A and 6B is a cabinet interior 92, which is depicted as the volume of space within cabinet 92. In an example, conditioned air is provided to the cabinet interior 92 to maintain the scan, system 18 at a set temperature and humidity, in an alternate embodiment conditioned air is provided to the isolation space 90 as a redundant layer of protection between the ambient zone 86 and the cabinet Interior 92. In one example, a conditioned air system (not shown) provides conditioned, air to the cabinet interior and to the isolation space 90. In an example, to avoid ingress of air into the cabinet interior 92 across sidewalls of the cabinet 19, pressure P₉₂ in the cabinet interior 92 is greater than a pressure in the isolation space 90. Similarly, pressure P₉₀ can be greater than pressure P₈₆ in the ambient zone 86. In one alternate example of operation, one system (not shown) supplies conditioned air to the cabinet interior 92, while another system (not shown) supplies conditioned air to the isolation space 90. Thus the Isolation space 90 provides not only a redundant way to protect the scan system 18 from damaging effects of warm or humid air, hut also provides a second or dual system tor providing conditioned air to the scan system 18. Moreover, the sidewalk of the scan trailer 12 and cabinet 19 can be thermal walls, that is, the side walls are insulated and have sufficient mass that they act as a thermal sink. The combination of moving seal within the radiation boundary satisfies the need of the scanning system and the sample to be at different but constant temperatures. Thus in the event the supply of conditioned air is lost, the temperature in the cabinet interior 92 can he maintained for a period of time longer than if the sidewalls did not include thermal walls,

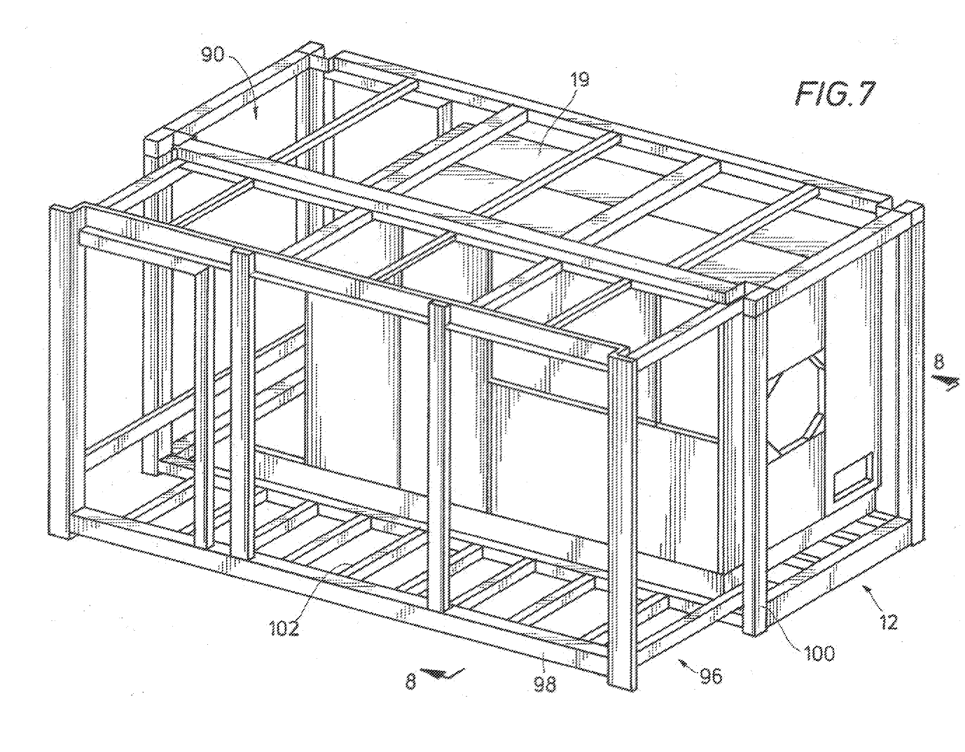

Referring now to FIG. 7, shown in perspective view Is an example of the scan trailer 12 having a popout portion 96 that projects laterally outward, thereby increasing the volume of the isolation space 90 within scan trailer 12. In the embodiment of FIG. 7, sidewalls of the scan trailer 12 are not shown thereby revealing the structural members making up the frame 98 of the popout 96 and enclosure frame 100 of the base portion of the scan trailer 12. Included with the frame 98 are stringers 102 that extend generally horizontally and provide a lower support for the inside of the expanded isolation space 90 when the popout portion 96 is the deployed configuration shown in FIG. 7. When in a retracted configuration, stringers 102 are disposed adjacent horizontal lower members making up the enclosure frame 100, but slide outward when the popout portion 96 is set into the deployed configuration. Popout portion 96 is adequately sealed so that a mobile clean room capability is provided therein, that is, the space with the popout portion 96 is generally free of dust and other contaminants that could alter or deleteriously affect operation of scanning equipment within or adjacent to the popout portion 96.

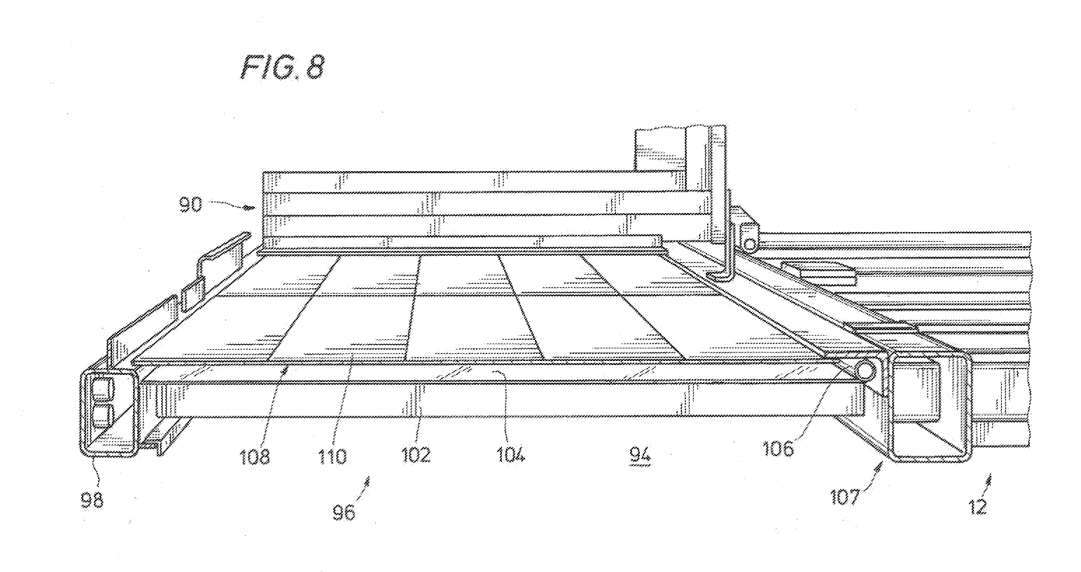

FIG. 8 is a sectional view of a portion of the scan trailer 12 and taken along lines 8-8 of FIG. 7. Here a membrane 104 is shown set across an upper surface of the stringers 102, which provides a way to block communication between the ambient, zone 94 outside of the scan trailer 12 and the enlarged portion of the isolation space 90 within the popout portion 96. In the example embodiment of FIG. 8, membrane 104, is a planar member, which can be made from a flexible material that blocks air flow, such as a fabric, a polymer, a composite layer, and the like, and combinations thereof. When in a retracted position, and the popout portion 96 is not deployed, membrane 104 can be rolled up onto a roller 106 shown, disposed adjacent a horizontal beam 107 of frame 100 intersected stringers 102. Flooring 108 is shown spaced upward from the membrane 104 to provide structural support on the lower portion of the popout portion 96. In an example, the flooring 108 is made up of segments 110 that tit together to form the flooring 108, In an example, the flooring 108 supports devices that may be deployed in the popout portion 96, as well as personnel that may in that section of the scan trailer 12.

Referring back to FIGS. 6A and 6B, shown in schematic view is an example of a conditioned air supply system 112 for supplying conditioned air to the isolation space 90 and cabinet Interior 92. Included with this example of the conditioned air supply system 112 is an air conditioner 114, which selectively conditions the air so that air being discharged from the air conditioner 114 is at a designated temperature, pressure, and humidity. In an embodiment, the air conditioner 114 includes a refrigeration unit that cools the air thereby removing moisture from supply sir, thereby achieving a designated humidity and/or temperature in air being discharged from the air conditioner 114. A fan or blower (not shown) may further be included with the air conditioner 114 for pressurizing the air being discharged from the air conditioner 114. Vent lines 118, 120 are illustrated for selectively delivering conditioned air to the cabinet interior 92 and the isolation space 90 respectively. Optional valves 122, 124 are shown disposed in lines 118, 120 respectively and for controlling a volume and pressure of air flow through lines 118, 120. A controller 126 is further optionally provided and that is in communication with conditions (i.e. temperature, humidity, and/or pressure) within the isolation space 90 and cabinet interior 92 via control lines 128, 130 that respectively tap into the Isolation space 90 and cabinet interior 92. In one example, feedback via control lines 128, 130 to the controller 126 can be used to control operation of the air conditioner 114 as well as selective operation of the valves 120, 122. An advantage of the conditioned air supply system 112 is that the air (or other fluid) being used to condition the isolation space 90 and cabinet interior 92 can be monitored and controlled to ensure an environment is maintained, at designated conditions to meet operating requirements for all hardware and components within the isolation space 90 and cabinet interior 92. Alternatively, pressure within the isolation space 90 and cabinet interior 92 can he controlled by virtue of the conditioned air supply system 112, so that in one example the cabinet interior 92 operates at a pressure greater than a pressure in the isolation space 90. The valves 122, 124 can be manipulated to create this pressure differential. Moreover, by Inclusion of the conditioned, air supply system 112, pressure in each of the isolation space 90 and cabinet interior 92 can be greater than a pressure of the environment ambient to the scan trailer 12.

The present invention described herein, therefore, is well adapted to carry out the objects and attain the ends and advantages mentioned, as well as others inherent therein. While a presently preferred embodiment of the invention has been given for purposes of disclosure, numerous changes exist in the details of procedures for accomplishing the desired results. For example, in an embodiment, mounting and shook absorption hardware is provided for securing the components in the core analysis system 10 to maintain their integrity and alignment during transportation in the trailers. The gantry can include reinforced mounting for rotating elements and added adhesive for hoard mounted components, e.g. integrated circuitry, resistors, capacitors, aid the like. A transport locking mechanism can he used to prevent sliding door movement when power is removed, and a locking mechanism can be used on ail threaded fasteners. All circuit boards can be mechanically secured to reduce vibration and remove gravity loading on connectors. Relays can be secured to mounting sockets, and expansion loops can be added in all cables and hoses and secured to cabinet walls. High voltage cables can be cushioned, and service door fastening can be added to prevent load on interlock closure. Cooling fan mounting can be reinforced and cooler unit can be secured for shipment. Also, transformer can be set near high voltage generator by mounting to the floor of the cabinet. An advantage of this is a scanned image of the core sample 24 can be produced at a resolution of up to 200 microns. These and other similar modifications will readily suggest themselves to those skilled in the art, and are intended to he encompassed within the spirit of the present invention disclosed herein and the scope of the appended claims.

What is claimed is:

1. A system for analyzing a core sample comprising:
a mobile enclosure;
a cabinet in the mobile enclosure having sidewalls which define a cabinet interior that is selectively at a higher pressure than an environment ambient to the mobile enclosure;
a core sample scan system within the cabinet; and
a loading assembly coupled to the core sample scan system and that selectively moves the core sample within the core sample scan system.

2. The system of claim 1, further comprising an isolation space within the mobile enclosure and having sidewalls that define a barrier, so that the isolation space is selectively at a higher pressure than the environment ambient to the mobile enclosure.

3. The system of claim 2, wherein the cabinet interior is selectively at a higher pressure than the isolation space.

4. The system of claim 1, wherein the core sample scan system comprises a core carrier that selectively receives the core sample and that is reciprocated within an annular sample enclosure.

5. The system of claim 4, wherein a space inside in the sample enclosure is in communication with the environment ambient to the mobile enclosure so that the sample enclosure defines a portion of a pressure barrier between the environment ambient to the mobile enclosure and the cabinet interior and maintains a thermal stability of the core sample scan system and of the core sample.

6. The system of claim 5, further comprising an isolation space within the mobile enclosure and that is between the cabinet interior and the space inside the sample enclosure, wherein the isolation space is selectively at a higher pressure than the environment ambient to the mobile enclosure.

7. The system of claim 4, further comprising a brush seal having a curved outer periphery that is in sealing contact with an inner surface of the sample enclosure and which defines a pressure barrier between the environment ambient to the mobile enclosure and the cabinet interim.

8. The system of claim 1, wherein the mobile enclosure comprises a popout section that selectively projects laterally outward into a deployed configuration, and in which defines a mobile clean room capability.

9. The system of claim 8, further comprising a membrane having an end coupled with the popout section and an opposite end mounted on a roller, so that when the popout section projects into the deployed configuration, the membrane unrolls from the roller and defines a pressure barrier between the environment ambient to the mobile enclosure and the cabinet interior.

10. The system of claim 8, further comprising a flooring section on a lower inside surface of the popout section comprising sections that are laid down when the popout section is the deployed configuration, wherein the flooring section defines a pressure barrier between the environment ambient to the mobile enclosure and the cabinet interior.

11. The system of claim 1, wherein the cabinet provides a dust barrier, shields radiation, and provides a stable thermal environment.

12. A system for analyzing a core sample comprising:
a mobile enclosure;
an isolation space within the mobile enclosure and having sidewalls that define a barrier, so that the isolation space is selectively at a higher pressure than the environment ambient to the mobile enclosure;
a cabinet in the mobile enclosure having sidewalls which define a cabinet interior that is selectively at a higher pressure than pressure in the isolation space;
an annular sample enclosure that projects into the cabinet;
a core sample scan system within the cabinet having a core carrier that selectively receives the core sample and that is reciprocated within the annular sample enclosure; and
a loading assembly coupled to the core sample scan system and that selectively moves the core sample within the core sample scan system.

13. The system of claim 12, wherein a space inside in the sample enclosure is in communication with the environment ambient to the mobile enclosure so that the sample enclosure defines a portion of a pressure barrier between the environment ambient to the mobile enclosure and the cabinet interior and maintains a thermal stability of the core sample scan system and of the core sample.

14. The system of claim 13, further comprising a brush seal that couples to and circumscribes the core carrier, the brush seal having a curved outer periphery that is in sealing contact with an inner surface of the sample enclosure and which defines a pressure barrier between the environment ambient to the mobile enclosure and the cabinet interior.

15. The system of claim 12, wherein the mobile enclosure comprises a popout section that selectively projects laterally outward into a deployed configuration and a membrane having an end coupled with the popout section and an opposite end mounted on a roller, so that when the popout section projects into the deployed configuration, the membrane unrolls from the roller and defines a pressure barrier between the environment ambient to the mobile enclosure and the cabinet interior, wherein the popout section defines a space having a mobile clean room capability.

16. The system of claim 15, further comprising a flooring section on a lower inside surface of the popout section comprising sections that are laid down when the popout section is the deployed configuration, wherein the flooring section defines a pressure barrier between the environment ambient to tie mobile enclosure and the cabinet interior.

17. A method of analyzing a core sample comprising:
inserting the core sample within a core scan system that is housed within a mobile enclosure; and
pressurizing a cabinet that encloses the core scan system to a pressure that exceeds a pressure of an environment that is ambient to the mobile enclosure.

18. The method of claim 17, further comprising forming an isolation space in the mobile enclosure that surround the cabinet and pressurizing the isolation space to a pressure that exceeds the pressure of the environment that is ambient to the mobile enclosure.

19. The method of claims 17, further comprising supplying a constant flow of conditioned air to the cabinet at designated conditions that satisfies operational requirements of components within the cabinet.
